(19) United States
(12) Patent Application Publication
(10) Pub. No.: US 2016/0015362 A1
(43) Pub. Date: Jan. 21, 2016

(54) INTRAVASCULAR DEVICES, SYSTEMS, AND METHODS HAVING MOTORS

(71) Applicants: Jesse Jones, Woburn, MA (US); Derek Bruce Eldredge, Tyngsboro, MA (US); Jason Sproul, Watertown, MA (US); Nathaniel Kemp, Concord, MA (US); Al Dunfee, Byfield, MA (US)

(72) Inventors: Jesse Jones, Woburn, MA (US); Derek Bruce Eldredge, Tyngsboro, MA (US); Jason Sproul, Watertown, MA (US); Nathaniel Kemp, Concord, MA (US); Al Dunfee, Byfield, MA (US)

(73) Assignee: VOLCANO CORPORATION, San Diego, CA (US)

(21) Appl. No.: 14/800,214

(22) Filed: Jul. 15, 2015

Related U.S. Application Data
(60) Provisional application No. 62/025,260, filed on Jul. 16, 2014.

(51) Int. Cl. A61B 8/12; A61B 8/00

(52) U.S. Cl. CPC A61B 8/12; A61B 8/445; A61B 8/4461; A61B 8/56

(57) ABSTRACT

Medical imaging devices and systems are provided. The medical imaging devices may include a transducer to transmit ultrasound signals (waves) and to receive the reflected ultrasound signals for imaging a vessel of interest. Embodiments may include disposing the transducer at a proximal end of the intravascular device and disposing a motor at a distal end of the intravascular device along with a rotatable acoustic mirror. The transducer may transmit and receive the ultrasound signals to and from the rotatable acoustic mirror via an acoustic lumen extending along the length of the device. Other embodiments may include disposing the transducer, the motor, and the rotatable acoustic mirror at the distal end of the medical imaging device. Further embodiments may include using a concentric layered structure to provide multiple conductors for transmitting signals to and receiving signals from components disposed at the distal end of the intravascular device.

### CROSS REFERENCE TO RELATED APPLICATIONS

The present application claims priority to and the benefit of the U.S. Provisional Patent Application No. 62/025,260, filed Jul. 16, 2014, which is hereby incorporated by reference in its entirety.

### FIELD OF DISCLOSURE

The present disclosure generally relates to intravascular devices, such as catheters and guide wires, used in clinical diagnostic and therapeutic procedures, including intravascular ultrasound (IVUS) procedures. These intravascular devices may include a transducer to transmit ultrasound signals (waves) and to receive the reflected ultrasound signals for imaging a vessel of interest. Embodiments of the present disclosure include disposing the transducer at a proximal end of the intravascular device and disposing a motor at a distal end of the intravascular device along with a rotatable acoustic mirror. In some instances, the transducer may transmit and receive the ultrasound signals to and from the rotatable acoustic mirror via an acoustic lumen extending along the length of the intravascular device. Other embodiments of the present disclosure include disposing the transducer, the motor, and the rotatable acoustic mirror at the distal end of the intravascular device. Further embodiments of the present disclosure include using a concentric layered structure to provide multiple conductors for transmitting signals to and receiving signals from components disposed at the distal end of the intravascular device.

### BACKGROUND

IVUS imaging procedures are widely used in interventional cardiology as a diagnostic tool for assessing a vessel, such as an artery, within the body of the patient to determine the need for treatment, to guide intervention, and/or to assess the effectiveness of administered treatment. An IVUS imaging system uses ultrasound echoes to form a cross-sectional image of the vessel of interest. Typically, IVUS imaging uses a transducer in an intravascular device to transmit ultrasound signals (waves) and to receive the reflected ultrasound signals via an electric cable. The transmitted ultrasound signals (often referred to as ultrasound pulses) pass easily through most tissues and blood, but they are partially reflected by discontinuities arising from tissue structures (such as the various layers of the vessel wall), red blood cells, and other features of interest. The IVUS imaging system, which is connected to the intravascular device by way of a patient interface module, processes the received ultrasound signals (often referred to as ultrasound echoes) to produce a cross-sectional image of the vessel proximate to where the transducer may be located.

The two types of intravascular devices in common use today are solid-state and rotational. A conventional solid-state intravascular device may use an array of transducers (typically 64) distributed in close proximity around a circumference of a sheath, the sheath being an outer layer of the catheter. Also, an acoustic-matching path conducive to ultrasound wave propagation may be formed between the transducer and the sheath. The transducers are connected to an electronic multiplexer circuit. The multiplexer circuit selects transducers from the array for transmitting ultrasound signals and receiving reflected ultrasound signals. By stepping through a sequence of transmit-receive transducer pairs, the solid-state intravascular device can synthesize the effect of a mechanically scanned transducer element, but without moving parts. Since there is no rotating mechanical element, the transducer array can be placed in closer contact with blood and vessel tissue with minimal risk of vessel trauma, and the solid-state scanner can be wired directly to the IVUS imaging system with a simple electrical cable and a standard detachable electrical connector. In general, the need to flush the catheter with saline or other contrast media to form the acoustic-matching path is avoided.

On the other hand, a conventional rotational intravascular device may include a flexible drive cable that continually rotates inside the sheath of the catheter inserted into the vessel of interest. The drive cable may have a transducer disposed at a distal end thereof. The transducer is typically oriented such that the ultrasound signals propagate generally perpendicular to an axis of the catheter. In the typical rotational catheter, the sheath may be filled with fluid (e.g., saline) to protect the vessel tissue from the rotating drive cable and transducer while permitting ultrasound signals to freely propagate from the transducer into the tissue and back. As the drive cable rotates (e.g., at 30 revolutions per second), the transducer is periodically excited with a high voltage pulse to emit a short burst of ultrasound. The ultrasound signals are emitted from the transducer, through the fluid-filled sheath and sheath wall, in a direction generally perpendicular to an axis of rotation of the drive cable (i.e., the axis of the IVUS catheter). The transducer then listens for returning ultrasound signals reflected from various tissue structures, and the IVUS imaging system assembles a two dimensional image of the vessel cross-section from a sequence of several hundred of these ultrasound pulse/echo acquisition sequences occurring during a single revolution of the drive cable and the transducer.

However, the images obtained by the conventional rotational catheters exhibit distortion caused due to non-uniform rotational distortion (NURD) experienced by the rotating drive cable. The distorted images are less effective at providing the required insight into the vessel condition. NURD may occur due to, for example, friction between the drive cable and the sheath that encloses the drive cable; friction between the sheath and the vessels through which the catheter travels through during use; non-symmetrical drive cable/transducer assembly that causes the drive cable to resist bending more at some angles than at other angles (when rotated, these asymmetries cause the drive cable to store more energy in some angular orientations and then to release that energy as the drive cable is rotated past that angle); the sheath and drive cable containing various bends and twists along its path to the vessel of interest, resulting in the transducer rotating at a non-uniform angular velocity even though one portion (e.g., the proximal portion) of the drive cable is rotated at a near-constant speed (because real actuators have limited torque, unlike ideal actuators). The use of a drive cable also contributes to a reduced track-ability and torque-ability as compared to non-rotational catheters, thereby rendering the intravascular device less easy to use. Further, inclusion of the drive cable undesirably leads to a larger diameter of the intravascular device which makes the device more difficult (or impossible) to deliver to all desired parts of the body. As such, the conventional rotational intravascular devices which include drive cables fail to adequately minimize NURD, lead to a less desirable design of the intravascular device, and contribute to additional cost, delay, and difficulty of imaging, diagnosing, or treating the patient.

Accordingly, there remains a need for improved ultrasound intravascular devices for use in IVUS imaging and associated devices, systems, and methods. The devices, systems, and methods proposed in the present disclosure overcome one or more of the deficiencies of conventional intravascular devices.

### SUMMARY

In one aspect, the present disclosure provides an intravascular ultrasound (IVUS) device including an acoustic lumen having a proximal end and a distal end, a transducer coupled to the acoustical lumen near the proximal end, and a mirror disposed near the distal end of the acoustic lumen, the mirror being able to rotate about a longitudinal axis of the IVUS device, wherein the acoustic lumen may enable communication of ultrasound signals between the transducer and the mirror. In some embodiments, the IVUS device may include a motor assembly disposed near the distal end of the acoustic lumen, wherein the mirror is fixedly attached to the motor assembly allowing the motor to rotate. In some embodiments, the motor may include a hollow shaft having a proximal opening coupled to the distal end of the acoustic lumen and a distal opening positioned adjacent to the mirror. In some embodiments, the mirror may be positioned adjacent to an opening at the distal end of the acoustic lumen. In some embodiments, a characteristic of the projected ultrasound signals may be varied based on a relationship between a frequency response of the transducer and a frequency response of the acoustic lumen. In some embodiments, the mirror may include a reflective surface that is configured to enable projection of the ultrasound signals from the distal end of the acoustic lumen towards the proximal end of the acoustic lumen.

In one aspect, the present disclosure provides intravascular ultrasound (IVUS) device having a proximal and a distal end. The IVUS device may include a transducer disposed near the distal end of the IVUS device, a motor assembly disposed near the distal end of the IVUS device, and a mirror fixedly attached to a rotating component of the motor assembly such that the mirror rotates about a longitudinal axis of the IVUS device with the rotating component of the motor assembly. The transducer and the mirror may be arranged to communicate ultrasound signals with each other. In some embodiments, the transducer may be stationary. In some embodiments, the transducer may be powered using a first cable and the motor is powered using a second cable. At least a portion of the first cable or the second cable is covered with a protective portion to limit interference with respect to the ultrasound signals. In some embodiments, the protective portion may be made of indium titanium oxide. In some embodiments, the transducer may be disposed distally of the motor assembly. In other embodiments, the transducer may be disposed proximately of the motor assembly. The transducer and the motor assembly may be disposed coaxially with respect to a longitudinal axis of the IVUS device.

In another aspect, the present disclosure provides an intravascular ultrasound (IVUS) device having a proximal end and a distal end. The IVUS device may include a transducer disposed near the distal end of the IVUS device, and a motor disposed near the distal end of the IVUS device. The transducer and the motor may be disposed coaxially with respect to a longitudinal axis of the IVUS device, and the transducer may be fixedly attached to a rotatable portion of the motor such that the transducer rotates with the rotatable portion. In some embodiments, the transducer may be communicatively coupled to a patient interface module located at the proximal end of the IVUS device using a conductor. In some embodiments, the conductor may at least partially include a concentric layered structure as part of the rotatable portion. The concentric layered structure may include alternating concentric layers of conductive and non-conductive material. In some embodiments, the conductor may be connected to at least one stationary cable. In some embodiments, the conductor may be connected to the at least one stationary cable using a slip ring configuration.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure. Throughout this description, like elements, in whatever embodiment described, refer to common elements wherever referred to and referenced by the same reference number. The characteristics, attributes, functions, interrelations ascribed to a particular element in one location apply to those elements when referred to by the same reference number in another location unless specifically stated otherwise.

The figures referenced below are drawn for ease of explanation of the basic teachings of the present disclosure only; the extensions of the figures with respect to number, position, relationship, and dimensions of the parts to form the following embodiments will be explained or will be within the skill of the art after the following description has been read and understood. Further, the exact dimensions and dimensional proportions to conform to specific force, weight, strength, and similar requirements will likewise be within the skill of the art after the following description has been read and understood.

The following is a brief description of each figure used to describe the present disclosure, and thus, is being presented for illustrative purposes only and should not be limitative of the scope of the present disclosure.

FIG. 1 illustrates an exemplary imaging system according to an embodiment of the present disclosure.

FIG. 2 illustrates a partial cutaway perspective view of an exemplary intravascular device according to an embodiment of the present disclosure.

FIG. 3 illustrates a block diagram of an exemplary patient interface module (PIM) according to an embodiment of the present disclosure.

FIG. 4 illustrates an exemplary configuration of an imaging system according to an embodiment of the present disclosure.

FIGS. 5A and 5B illustrate cross-sectional side views of exemplary intravascular devices according to embodiments of the disclosure.

FIG. 6A illustrates a cross-sectional end view of an exemplary rotor shaft according to an embodiment of the present disclosure.

FIG. 6B illustrates a cross-sectional side view of an implementation of the rotor shaft illustrated in FIG. 6A according to an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to some embodiments may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

As discussed above, there remains a need for improved ultrasound intravascular devices to be used in IVUS imaging procedures and associated devices, systems, and methods. The present disclosure describes devices, systems, and methods to eliminate non-uniform rotational distortion caused due to a drive cable and to reduce the outer diameters of the intravascular devices. In particular, the present disclosure proposes intravascular devices that include acoustic lumens instead of drive cables. In some embodiments of the disclosed intravascular device, the transducer may be disposed at a proximal end of the intravascular device and a motor along with a rotatable acoustic mirror may be disposed at a distal end of the intravascular device. The transducer may transmit and receive the ultrasound signals to and from the rotatable acoustic mirror via an acoustic lumen included in the intravascular device. Other embodiments of the present disclosure include disposing the transducer, the motor, and the rotatable acoustic at the distal end of the intravascular device. Further embodiments of the present disclosure include using a concentric layered structure of the motor shaft to provide multiple conductors for transmitting to and receiving signals from components disposed at the distal end of the intravascular device.

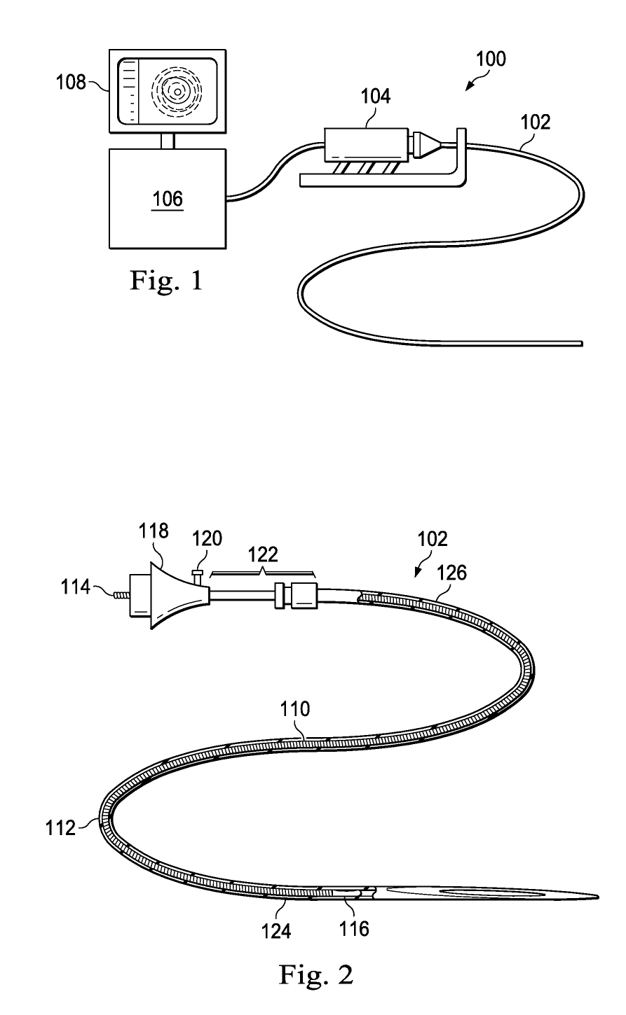

FIG. 1 illustrates an exemplary IVUS imaging system 100 according to an embodiment of the present disclosure. The IVUS system may utilize any type of suitable IVUS imaging device, including rotational devices. In some particular embodiments, the present disclosure may incorporate a transducer. The transducer may be a piezoelectric micromachined ultrasound transducer (PMUT), a piezoelectric zirconate transducer (PZT), or a capacitive micromachined ultrasonic transducer (CMUT). In some embodiments of the present disclosure, the IVUS imaging system 100 may be a rotational IVUS imaging system including a rotatable acoustic mirror (described below). In that regard, the main components of the rotational IVUS imaging system may include an IVUS device 102, a patient interface module (PIM) 104, an IVUS console or processing system 106, and a monitor 108 to display the IVUS images provided by the IVUS console 106. Generally, the intravascular device 102 may be configured to take on any desired arcuate profile when in the curved configuration. The intravascular device 102 is sized and shaped to be inserted into a vessel of a patient's body. In some instances, the intravascular device 102 may have an external diameter ranging from 0.014 inches to 0.108 inches. As discussed in greater detail below, the rotational IVUS intravascular device 102 may include a transducer along with its associated circuitry mounted near a proximal end of the intravascular device 102 or near a distal end of the intravascular device 102. The PIM 104 may allow delivery of DC supply voltages to the transducer circuitry. In that regard, in some embodiments, the transducer may be included within the PIM 104. The PIM 104 may generate and/or provide the required sequence of transmit trigger signals and control waveforms to regulate the operation of the circuitry, and may process the amplified echo signals received over, for example, that same conductor pair. The PIM 104 may also supply the high- and low-voltage DC power supplies to support operation of the rotational components of the IVUS intravascular device 102.

FIG. 2 illustrates a diagrammatic, partial cutaway perspective view of the exemplary intravascular device 102 according to an embodiment of the present disclosure. In that regard, FIG. 2 shows additional detail regarding the structural features of the intravascular device 102. In some respects, the intravascular device 102 may be similar to traditional rotational IVUS devices, such as the Revolution® catheter available from Volcano Corporation and described in U.S. Pat. No. 8,104,479, or those disclosed in U.S. Pat. Nos. 5,243,988 and 5,546,948, each of which is hereby incorporated by reference in its entirety. However, the intravascular device 102 according to the present disclosure may include an acoustic lumen instead of the conventional drive cable. In that regard, the rotational intravascular device 102 may include an acoustic lumen 110 and an outer catheter/sheath assembly 112. An acoustic lumen may be formed of two materials used to conduct ultrasound imaging with minimal loss or distortion. The first inner material may be a fluid or gel that allows the ultrasound waves to propagate. The second outer material, typically a polymer, may contain the inner material and limit the ultrasound energy from escaping from the inner material. The acoustic lumen 110 is flexible and terminates at the proximal end of the intravascular device 102. Further, a portion 114 of the acoustic lumen 110 may be in communication with a transducer 300 included in the PIM 104. As such, the acoustic lumen 110 may at least partially be enclosed in the catheter/sheath assembly 112. The distal end of the flexible acoustic lumen 110 may be in communication with a rotatable acoustic mirror. In that regard, a motor assembly 116 containing a motor and associated rotor may be connected to the rotatable acoustic mirror and configured to rotate the rotatable acoustic mirror to scan the ultrasound signals across the vasculature, as described in greater detail below.

The catheter/sheath assembly 112 may include a hub 118 to interface with the PIM 104 and may provide a bearing surface and a fluid seal between the elements of the intravascular device. The hub 118 may include a luer lock flush port 120 through which saline may be injected to flush out the air and fill the sheath 112 and/or acoustic lumen 110 of the intravascular device 102 with an ultrasound-compatible fluid at the time of use. The saline or other similar flush may be typically required since air does not readily conduct ultrasound. Saline also provides a biocompatible lubricant for any enclosed components. The hub 118 may be coupled to a telescope 122 that includes nested tubular elements and a sliding fluid seal that permit the catheter/sheath assembly 112 to be lengthened or shortened to facilitate axial movement of the motor assembly 116 within an acoustically transparent window 124 of the distal portion of the intravascular device 102. In some embodiments, the window 124 may include thin-walled plastic tubing fabricated from material(s) that readily conduct ultrasound waves between the acoustic mirror and the vessel tissue with minimal attenuation, reflection, or refraction. A proximal portion 126 of the catheter/sheath assembly 112 may bridge the catheter segment between the telescope 122 and the window 124, and may include a material or composite to provide a lubricious internal surface and optimum stiffness, but without the need to conduct ultrasound.

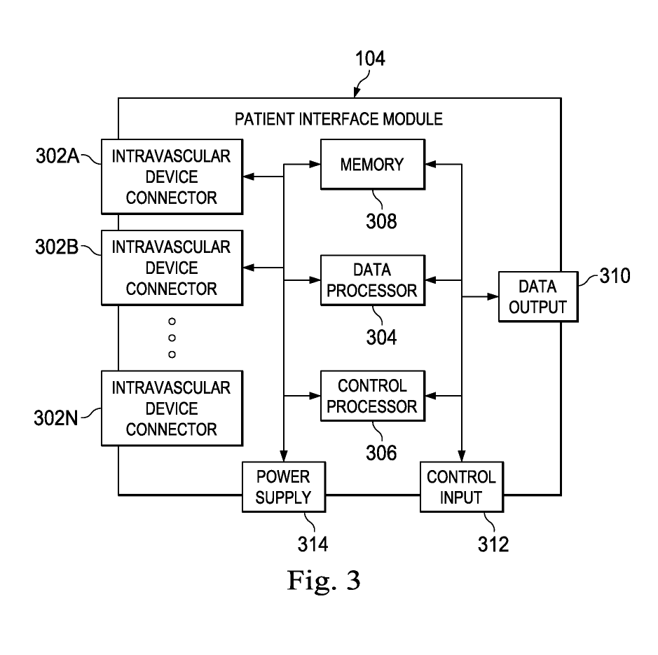

FIG. 3 illustrates components included in the exemplary PIM 104 according to an embodiment of the present disclosure. The PIM 104 may include intravascular device connectors 302A-N, a data processor 304, a control processor 306, a memory 308, a data output port 310, a control input port 312, and a power supply port 314. In the illustrated embodiment, the PIM 104 may include a plurality of intravascular device connectors to interface with one or more intravascular devices 102 at a given time. In some embodiments, only one of the intravascular device connectors 302 may be operational to communicate with an intravascular device 102. For example, in the illustrated embodiment, intravascular device connector 302A includes the transducer 300.

Each of the intravascular device connectors 302A-N may be in communication with a data processor 304 and a control processor 306. In general the PIM 104 may include one or more processors, with some processors being allocated specific tasks or with tasks being distributed among the processors. In some embodiments, the one or more processors, including data processor 304 and control processor 306 may be implemented as a configuration of a field-programmable gate array (FPGA), a programmable logic device (PLD), or another programmable processor. In some embodiments, the data processor 304 and the control processor 306 may be provided by a single processor. In embodiments of the PIM 104 that include a programmable processor, one or more configurations of the processor may be stored in a memory 308, from which they can be implemented as needed.

The memory 308 may be a hard disk drive, a solid-state drive, RAM, or other type of memory device that can hold instructions and/or data. The memory 308 may be a volatile or a non-volatile memory. Data may be gathered from an intravascular device 102 connected through the intravascular device connector 302A to the data processor 304 and/or the control processor 306, which may store some or all of that data in memory 308 before, during, and/or after data or signal processing is performed by the data processor 304. After signal processing is performed on the data in memory 308, the control processor 306 may cause the processed data to be transmitted from memory 308 to an external device such as the console 106, discussed above with respect to FIG. 1, through a data output port 310. The data output port 310 in some embodiments may be a fiber optic data output including a small form factor pluggable transceiver. However, not all embodiments of the intravascular device interface 300 include a fiber optic data output for the data output port 310.

In addition to outputting data through the data output port 310, the PIM 104 may be configured to receive control instructions and queries through a control input port 312. Depending on the particular embodiment of the PIM 104, the control input port 312 may be configured to interface with a handpiece controller, a controller provided in communication with a console such as a keyboard, a mouse, or a touch screen display, or from another controller. The PIM 104 may further include a power supply 314 that is configured to supply power internally to the PIM 104, e.g. to components including the data processor 304, the control processor 306, the memory 308, and other components. Power supply 314 may further be configured to provide power through the intravascular device connectors 302A-N to any and all components associated with the intravascular devices coupled thereto.

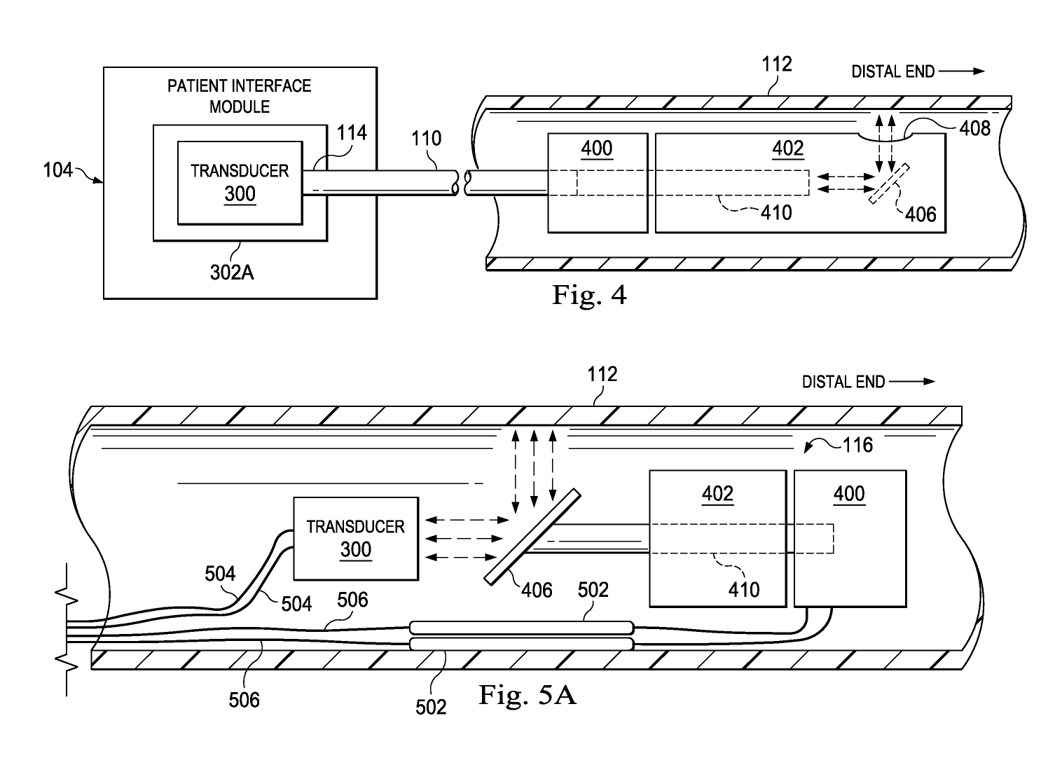

FIG. 4 illustrates an interface between the exemplary PIM 104 and the motor assembly 116 according to an embodiment of the present disclosure. The motor assembly 116 may include a stationary component (e.g., a stator) 400 and a rotatable component (e.g., a rotor) 402, the rotatable component 402 including a fixedly attached acoustic mirror 406 and an ultrasound-compatible opening 408. The motor assembly 116 may be powered using conductors extending from the PIM 104 to the motor assembly 116. In some embodiments, the acoustic lumen 110 and the motor assembly 116 may be disposed coaxially with respect to a longitudinal axis of the intravascular device 102. The stationary component 400 and the rotatable component 402 may include a hollow shaft 410 therebetween. As illustrated, a proximal opening of the hollow shaft 410 may be coupled to the acoustic lumen 110. Further, a distal opening of the hollow shaft 410 may be positioned adjacent to the reflective surface of the acoustic mirror 406. This configuration allows the ultrasound signals to propagate between the transducer 300 and the reflective surface of the acoustic mirror 406. For example, the ultrasound signals and corresponding echo signals may be propagated from the transducer 300, through the acoustic lumen 110, and the hollow shaft 410, to the reflective surface of the acoustic mirror 406, and vice versa.

The motor assembly 116 may be secured in place relative to the intravascular device 102 by an epoxy 148 or other bonding agent, while allowing the sheath of the intravascular device 102 to be filled with ultrasound-compatible fluid. Other options to secure the motor assembly 116 may be used. These options include forcing the motor assembly 116 into an interference-fit with a surrounding tubing or structure, laser welding or soldering to metal structures in the surrounding device, or forming one of the other components of the device by overmolding it around the motor assembly 116 (the motor assembly 116 having recessed and/or protruding portions that interlock with the flowing overmold polymer to solidify into a bond joint). In an alternate embodiment, the acoustic mirror 406 and the ultrasound-compatible opening 408 may be positioned closer to the distal opening of the acoustic lumen 110 than the stationary component 400, thereby eliminating the need for the hollow driveshaft 410. Finally, the sheath of the intravascular device 102 may be filled with an ultrasound-compatible fluid (e.g., saline) to facilitate the IVUS imaging procedure.

During the IVUS imaging procedure, the transducer 300 may project the ultrasound signals into the portion 114 of the acoustic lumen 110 in communication with the transducer 300. The acoustic lumen 110 may then carry the projected ultrasound signals down to the hollow shaft 410, through which the ultrasound signals may reach the reflective surface of the acoustic mirror 406. The rotatable component 402, and therefore the acoustic mirror 406, may be rotated about an axis of the intravascular device 102 at a desired angular velocity. This allows the ultrasound signals to propagate perpendicular to the axis of the intravascular device 102 through the ultrasound compatible opening 408. In other words, as the acoustic mirror 406 rotates, the ultrasound signals may be projected from the reflective surface of the acoustic mirror 406, through the opening 408, the ultrasound-compatible fluid, and the sheath wall 112, in a direction generally perpendicular to an axis of rotation of the rotatable component 402 (i.e., perpendicular to the longitudinal axis of the IVUS catheter). The returning ultrasound signals reflected from various tissue structures may be captured by the acoustic mirror 406, and projected towards the distal opening of the hollow shaft 410. These returning ultrasound signals may then be carried to the transducer 300 through the hollow shaft 410 and the acoustic lumen 110. The PIM 104 may then assemble a multi-dimensional image of the vessel cross-section based on a sequence of several hundred of these returning ultrasound pulse/echo signals received during a single revolution of the acoustic mirror 406. It should be noted that the acoustic mirror 406 may be mounted at an oblique angle with respect to the longitudinal axis of the intravascular device 102, which can be used to obtain flow data in addition to imaging data as described in U.S. patent application Ser. No. 13/892,062, filed May 10, 2013 published as U.S. Patent Publication No. 2013/0303920 on Nov. 14, 2013, which is herein incorporated by reference in its entirety.

In this way, by using the acoustic lumen 110 and placing the motor assembly 116 along with a rotatable mirror 406 at the distal end of the intravascular device 102, the IVUS images may be generated without using a rotating drive cable extending the length of the intravascular device 102. This allows for a simplified design of the IVUS imaging system. For example, the embodiments of the present disclosure include fewer moving parts and minimize the effect of NURD on the IVUS images. Also, since there is no drive cable, the outer diameter of the intravascular device 102 can be smaller in comparison to the conventional intravascular devices. In addition, the design is further simplified due to the incorporation of the transducer 300 within the PIM 104 at the proximal end because all the circuitry and the electrical wiring associated the transducer 300 remains contained within the PIM 104. Additional advantages may be observed with the use of the acoustic lumen 110. In particular, a characteristic (e.g., an intensity) of the ultrasound signals being propagated through the acoustic lumen 110 may be manipulated or varied based on a frequency response of the acoustic lumen 110 and/or the frequency response of the transducer 300. For example, the frequency response of the acoustic lumen 110 and/or the frequency response of the transducer 300 may be tuned with respect to each other to either operate on-resonance or off-resonance, thereby manipulating the ultrasound signals.

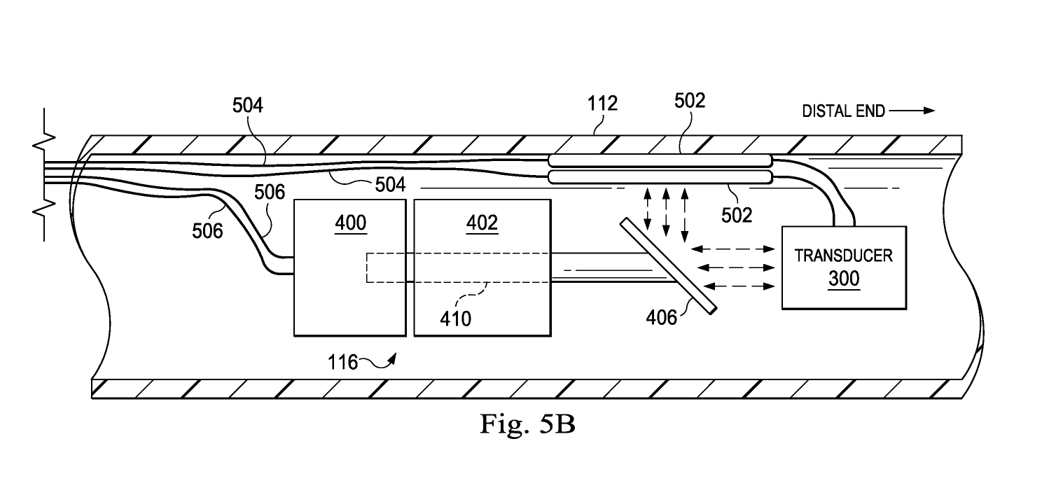

FIG. 5A illustrates a cross-sectional side view of the exemplary intravascular device 102 according to an embodiment of the present disclosure. In some embodiments, the intravascular device 102 is implemented without a drive cable or an acoustic lumen, and with the transducer 300 disposed near the distal end of the intravascular device 102. The transducer 300 and the motor assembly 116 may be disposed coaxially with respect to a longitudinal axis of the intravascular device 102. The intravascular device 102 may include the transducer 300 and the motor assembly 116 disposed near the distal end of the intravascular device 102. The motor assembly 116 may include the stationary component 400 and the rotatable component 402 with the hollow shaft 410 connected to the acoustic mirror 406. In some embodiments, the transducer 300 may be located distally, i.e., towards the distal end of the intravascular device 102, with respect to the motor assembly 116 such that the acoustic mirror 406 is proximal of the stationary component 400 and the transducer 300 is distal of the stationary component 400. Also, the transducer 300 and a reflective surface of the mirror 406 may be positioned to face each other, thereby allowing communication of ultrasound signals directly between the transducer 300 and the reflective surface of the mirror 406 without the use of the acoustic lumen 110 or the hollow shaft 410 (e.g., as shown in FIG. 5B).

The motor assembly 116 may be powered using cables 506 and the transducer may be powered using cables 504. The cables 504, 506 may be connected to the PIM 104 and may be implemented in the form of thin conductive wires, which may be formed of copper, gold, silver, or other suitable materials. In some embodiments, at least the cables 504 may be configured to communicate data associated with ultrasound signals between the transducer 300 and the PIM 104. In some embodiments, at least a portion of either one or both of the cables 504, 506 may include a protective indium-tin-oxide (ITO) portion 502 in place of a portion of the conductive metallic wires. This minimizes the effects of “shadowing” caused due to interference of the conductive metallic wires with the ultrasound signals. Since the ITO portion 502 is ultrasound compatible, it interferes less with the ultrasound signals as compared to the conductive metallic wires. The ITO portion 502 may be over-coated on one or both sides with an electrical insulator as needed to further prevent any interference or instances of short-circuits through the surrounding fluids. In that regard, even if the ITO portion 502 has a relatively high acoustic impedance with respect to the surrounding materials and components, any disturbance or discontinuity introduced by the same may be accommodated easily during signal and image post-processing.

FIG. 5B illustrates a cross-sectional side view of an exemplary intravascular device 102 according to an embodiment of the present disclosure. The intravascular device 102 illustrated in FIG. 5B is similar to the intravascular device 102 illustrated in FIG. 5A in that the intravascular device 102 illustrated in FIG. 5B may be implemented without a drive cable or an acoustic lumen, and with the transducer 300 disposed at the distal end of the intravascular device 102. Also, the intravascular device 102 illustrated in FIG. 5B may similarly include the transducer 300 and the motor assembly 116 including the stationary component 400 and the rotatable component 402 with the hollow shaft 410 connected to the acoustic mirror 406 near the distal end of the intravascular device 102. The motor assembly 116 may be similarly powered using cables 506 and the transducer may be powered using cables 504. Further, the cables 504, 506 may be connected to the PIM 104 and may be implemented in the form of thin conductive wires, which may be formed of copper, gold, silver, or other suitable materials. In some embodiments, as discussed above, at least a portion of either one or both of the cables 504, 506 may include a protective indium-tin-oxide (ITO) portion 502 in place of a portion of the conductive metallic wires. Since the ITO portion 502 is ultrasound compatible, it does not interfere with the ultrasound signals and also limits the interference of the conductive wires with respect to the ultrasound signals. The ITO portion 502 may be over-coated on one or both sides of the conductors with an electrical insulator as needed to prevent any instances of short-circuits through the surrounding fluids. In that regard, even if the ITO portion 502 has relatively high acoustic impedance with respect to the surrounding materials and components, any disturbance or discontinuity introduced by the same may be accommodated easily during signal and image post-processing. However, the configuration of the transducer 300 and the motor assembly 116 in the intravascular device 102 illustrated in FIG. 5B may be different in that the transducer 300 may be located proximally, i.e., towards the proximal end of the intravascular device 102, with respect to the motor assembly 116.

With respect to FIGS. 5A and 5B, during the IVUS procedure, the transducer 300 may project the ultrasound signals towards the reflective surface of the acoustic mirror 406. The rotatable component 402, and therefore the acoustic mirror 406, may be rotated about an axis of the intravascular device 102 at a desired angular velocity. This allows the ultrasound signals to propagate perpendicular to the axis of the intravascular device 102. In other words, as the acoustic mirror 406 rotates, the ultrasound signals may be projected from the reflective surface of the acoustic mirror 406, through the opening 408, the ultrasound-compatible fluid, and the sheath wall 112, in a direction generally perpendicular to an axis of rotation of the rotatable component 402 (i.e., perpendicular to the longitudinal axis of the IVUS catheter). The returning ultrasound signals reflected from various tissue structures may be captured by the acoustic mirror 406, and projected towards the distal opening of the hollow shaft 410. These returning ultrasound signals may then be carried to the transducer 300 through the hollow shaft 410 and the acoustic lumen 110. The PIM 104 may then assemble a multi-dimensional image of the vessel cross-section based on a sequence of several hundred of these returning ultrasound pulse/echo signals received during a single revolution of the acoustic mirror 406. It should be noted that the acoustic mirror 406 may be mounted at an oblique angle with respect to the longitudinal axis of the intravascular device 102, which can be used to obtain flow data in addition to imaging data as described in U.S. patent application Ser. No. 13/892,062, filed May 10, 2013 published as U.S. Patent Publication No. 2013/0303920 on Nov. 14, 2013, which is herein incorporated by reference in its entirety.

In this way, by placing the transducer 300 and the motor assembly 116 with the rotatable acoustic mirror at the distal end of the intravascular device 102, the IVUS images may be generated without using a rotating drive cable or an acoustic lumen extending along the length of the intravascular device 102. This allows for a simplified design of the IVUS imaging system. For example, the embodiments of the present disclosure include fewer moving parts and minimize the effect of NURD on the IVUS images. Also, since there is no drive cable or an acoustic lumen, the outer diameter of the intravascular device 102 is smaller in comparison to the conventional intravascular devices, thereby limiting any trauma experienced by the patient. Additional advantages may be observed by eliminating the drive cable and the acoustic lumen from the design. Specifically, the intravascular device may be more flexible and may have better maneuverability within the body of the patient. This also allows for reduced trauma to the patient during the IVUS imaging procedure.

Now, as discussed above, the ITO portion 502 may have a relatively high acoustic impedance with respect to the surrounding materials and components, and any disturbance or discontinuity introduced in the IVUS images by the same may easily be accommodated during signal and image post-processing. However, this post-processing accommodation may be avoided. In particular, the conductive cables/protective portions used to form the plurality of electrical conducting paths may be arranged in a concentric layered structure within concentric layers of insulating material. In some embodiments, the concentric layered structure may be used in the vicinity of the transducer where the effects of “shadowing” may be observed. In this way, by using such rotationally symmetric structures such as the rotor shaft 600 instead of the wiring cables and the ITO portions, the need to accommodate any disturbance or discontinuity during post-processing may be avoided.

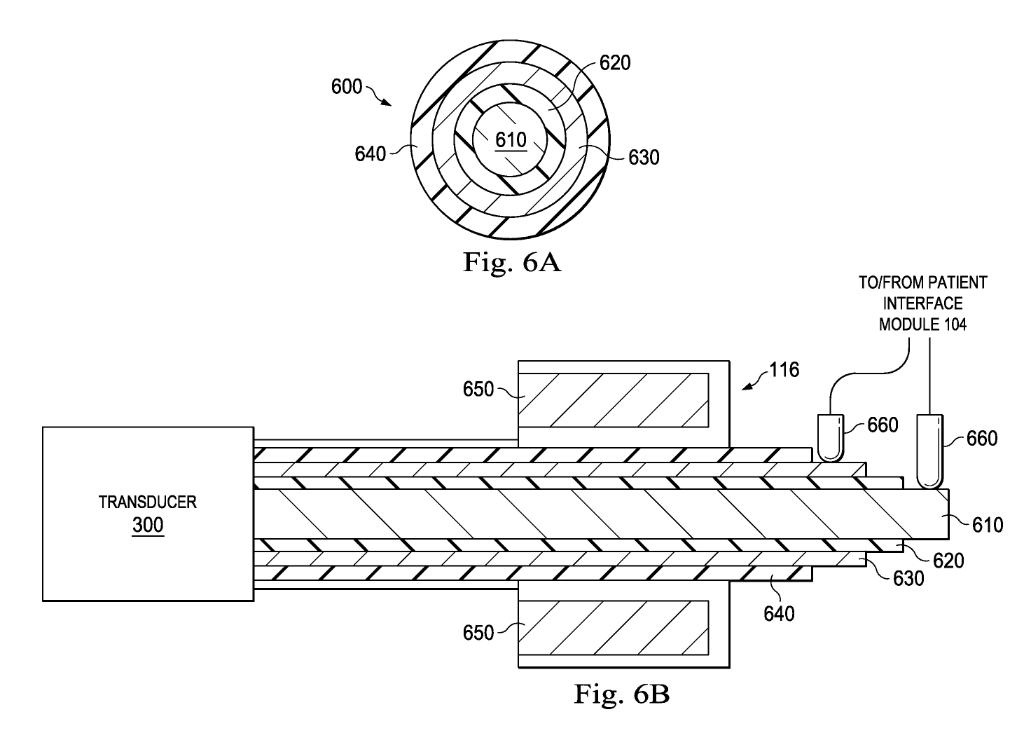

FIG. 6A illustrates a cross-sectional end view of a rotor shaft 600 according to an embodiment of the present disclosure. In some embodiments, the rotor shaft 600 may have a concentric layered structure including a conductive solid core 610, an insulator/dielectric layer 620, a conductive layer 630, and an insulator/dielectric layer 640. The conductive layers such as the solid core 610 and the conductive layer 630 may be used to provide power and/or data communication connections to the transducer, as discussed in further detail with respect to FIG. 6B. The non-conductive layers such as the insulator/dielectric layer 620, 640 may be used to provide insulation and/or isolation between the conductive layers 610, 630, as shown. Even though four concentric layers are shown in the illustrated embodiment, it should be appreciated that implementation of any number of concentric layers is within the scope of the present disclosure.

FIG. 6B illustrates a cross-sectional side view of an implementation of the rotor shaft 600 according to an embodiment of the present disclosure. In some embodiments, the rotor shaft 600 may be implemented at the distal end of the intravascular device 102. In some embodiments, the transducer 300 may be fixedly secured to the motor shaft 600 of the motor assembly 116. The electromagnets 650 of the motor assembly 116 may be powered to rotate the rotor shaft 600 about the longitudinal axis of the intravascular device 102, and thereby allow the fixedly secured transducer 300 to rotate in accordance with the rotation of the rotor shaft 600.

The rotor shaft 600 may be implemented within the motor assembly 116. In that regard, the rotor shaft 600 including the layered conductors 610, 620, 630, 640 may provide electrical connections to the transducer 300. The conductive layers 610, 630 may respectively be connected to electrical conductors 660 that provide electrical and data communication to and from the PIM 104 located at the proximal end of the intravascular device 102. For example, the PIM 104 generates and/or provides the required sequence of transmit trigger signals and control waveforms to regulate the operation of the circuit and processes the amplified echo signals received over electrical conductors 660. The transducer 300 is typically oriented such that the ultrasound signals propagate generally perpendicular to an axis of the intravascular device 102. In that regard, the transducer 300 may be oriented at an oblique angle with respect to the longitudinal axis of the intravascular device 102. The electrical conductors 660 may be implemented using a slip-ring structure to provide the transition between stationary components (e.g., conductors 660) located at the proximal end of the intravascular device 102 and the rotating components (e.g., transducer 300 and/or the rotor shaft 600) located at the distal end of the intravascular device 102. In some embodiments, a portion of one or more of the plurality of layers of the rotor shaft 600 may be removed to accommodate strengthening members or other features without affecting the insulating layers 620, 640 or the electrical or data connections through the conductive layers 610, 630.

It should be appreciated that while the exemplary embodiment is described in terms of an IVUS device, the present disclosure is not so limited. Thus, for example, other invasive medical devices such as, by way of non-limiting example, catheters, guidewires, and probes, having one or more sensing elements may utilize a similar approach to the mount the sensing element(s) and/or associated control circuitry. For example, in some instances pressure-sensing and/or flow-sensing intravascular devices utilize a similar approach in accordance with the present disclosure. Further, the devices, methods, and associated techniques discussed in present disclosure are not limited to intravascular imaging devices, and may be applied, for example, to other medical imaging devices including extra-vascular (extra corporeal) ultrasound devices and also to intracardiac or endoscopic devices.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A medical imaging device, comprising:
an acoustic lumen having a proximal end and a distal end;
a transducer coupled to the acoustical lumen near the proximal end; and
a mirror disposed near the distal end of the acoustic lumen, the mirror being configured to rotate about a longitudinal axis of the medical imaging device, wherein
the acoustic lumen is configured to enable communication of ultrasound signals between the transducer and the mirror.

2. The medical imaging device of claim 1, further comprising:
a motor assembly disposed near the distal end of the acoustic lumen, wherein the mirror is fixedly attached to the motor assembly allowing the mirror to rotate.

3. The medical imaging device of claim 2, wherein the motor comprises a stationary component and a rotatable component, the mirror being fixedly attached to the rotatable component.

4. The medical imaging device of claim 3, wherein the motor comprises a hollow shaft having a proximal opening coupled to the distal end of the acoustic lumen and a distal opening positioned adjacent to the mirror.

5. The medical imaging device of claim 2, wherein the mirror is positioned adjacent to an opening at the distal end of the acoustic lumen.

6. The medical imaging device of claim 1, wherein a characteristic of the ultrasound signals is varied based on a relationship between a frequency response of the transducer and a frequency response of the acoustic lumen.

7. The medical imaging device of claim 1, wherein the mirror includes a reflective surface configured to enable projection of the ultrasound signals from the distal end of the acoustic lumen towards the proximal end of the acoustic lumen.

8. A medical imaging device having a proximal end and a distal end, the medical imaging device comprising:
a transducer disposed near the distal end of the medical imaging device;
a motor assembly disposed near the distal end of the medical imaging device; and
a mirror fixedly attached to a rotating component of the motor assembly such that the mirror rotates about a longitudinal axis of the medical imaging device with the rotating component of the motor assembly, wherein
the transducer and the mirror are arranged to communicate ultrasound signals between each other.

9. The medical imaging device of claim 8, wherein the transducer is stationary.

10. The medical imaging device of claim 8, wherein the transducer is powered using a first cable and the motor is powered using a second cable.

11. The medical imaging device of claim 10, wherein at least a portion of the first cable or the second cable includes a protective portion to limit interference with respect to the ultrasound signals.

12. The medical imaging device of claim 11, wherein the protective portion is made of indium titanium oxide.

13. The medical imaging device of claim 8, wherein the transducer is disposed distally of the motor assembly.

14. The medical imaging device of claim 8, wherein the transducer is disposed proximally of the motor assembly.

15. The medical imaging device of claim 8, when in the transducer and the motor assembly are disposed coaxially with respect to a longitudinal axis of the IVUS device.

16. A medical imaging device having a proximal end and a distal end, the IVUS device comprising:
a transducer disposed near the distal end of the medical imaging device; and
a motor disposed near the distal end of the medical imaging device, wherein
the transducer and the motor are disposed coaxially with respect to a longitudinal axis of the medical imaging device, and the transducer is fixedly attached to a rotatable portion of the motor such that the transducer rotates with the rotatable portion.

17. The medical imaging device of claim 16, wherein the transducer is communicatively coupled to a patient interface module located at the proximal end of the medical imaging device using a conductor.

18. The medical imaging device of claim 17, wherein the conductor includes a concentric layered structure.

19. The medical imaging device of claim 17, wherein the concentric layered structure includes alternating concentric layers of conductive and non-conductive material.

20. The medical imaging device of claim 17, wherein the motor assembly partially includes the conductor having a concentric layered structure as part of the rotatable portion.

21. The medical imaging device of claim 16, wherein the conductor is connected to at least one stationary cable.

22. The medical imaging device of claim 21, wherein the conductor is connected to the at least one stationary cable using a slip ring configuration.
